(19) United States
(12) Patent Application Publication
(10) Pub. No.: US 2008/0241204 A1
(43) Pub. Date: Oct. 2, 2008

(54) WETTING SOLUTION FOR WET WIPES FOR COSMETIC, PERSONAL HYGIENE, DERMATOLOGICAL AND/OR CLEANING PURPOSES

(75) Inventor: Ulrich Leikauf, Bammental (DE)

Correspondence Address: KNOBBE MARTENS OLSON & BEAR LLP, 2040 MAIN STREET, FOURTEENTH FLOOR, IRVINE, CA, 92614, US

(73) Assignee: ZSCHIMMER & SCHWARZ GMBH & CO. KG, Lahnstein (DE)

(21) Appl. No.: 12/060,018

(22) Filed: Mar. 31, 2008

(30) Foreign Application Priority Data
Mar. 31, 2007 (EP) 07 006 765.7

(51) Int. Cl. A61K 8/30; A61K 8/02; C11D 7/22

(52) U.S. Cl. 424/402; 514/772; 514/785; 514/783; 510/136; 510/159

(57) ABSTRACT

The present invention relates to a wetting solution for manufacturing wet wipes or other wet flexible materials, and to such wet wipes for cosmetic, body care, dermatological and/or cleaning purposes, such as cleaning wipes, facial cleaning wipes, make-up remover wipes, refreshment wipes, baby wipes, wet toilet paper or eyeglass cleaning wipes, wherein the preferably aqueous wetting solution contains at least one amino acid ester, preferably from the group of pyrrolidone carboxylic acid esters, as an emulsifier. The invention further relates to the use of such amino acid esters as emulsifiers in wetting solutions for manufacturing said wet tissues.

### CROSS-REFERENCE TO RELATED APPLICATIONS

This application claims the benefit of EP application No. 07 006 765.7, filed Mar. 31, 2007.

### BACKGROUND OF THE INVENTION

1. Field of the Invention

The present invention relates to wetting solutions for wet wipes for cosmetic, personal hygiene, dermatological and/or cleaning purposes, wherein the preferably aqueous solution contains at least one amino acid ester as an emulsifier. Further, the invention relates to the use of such amino acid esters as emulsifiers in manufacturing such wet wipes.

2. Description of the Related Art

Such tissues or wipes, respectively, find use as objects for everyday use in a wide range of applications, e.g. as cleaning wipes, facial cleaning wipes, makeup remover wipes, refreshment wipes, baby wipes, wet toilet paper, eyeglass cleaning wipes, and other wet wipes. These wipes allow cleaning or care, for example, of the skin, without the need for running water. This is also interesting in geriatric care in which the otherwise typical bathroom facilities cannot always be used. The wipes themselves, wetted with a solution, usually consist of paper or other natural or synthetic fibers, as described in EP 1029977 or U.S. Pat. No. 6,338,855, for example.

For cloths or tissues made of natural or synthetic fibers the wetting solution is often very diluted. Rather, additives with low water content are preferred for tissue papers. Conventional impregnating or wetting solutions, respectively, often contain polyethylene glycols and their derivatives as emulsifiers. For example, WO 95/35411 proposes lotions for wet wipes that contain, beside mineral oil, fatty acid esters, fatty alcohol ethoxylates and fatty alcohols. The majority of cosmetic wetting solutions are prepared according to the so-called phase inversion temperature process (PIT process, see also Shinoda K, Kunieda H, Encycl. of Emulsion Technology, pp. 337-367, 1983), as for example also proposed in WO 00/04230.

There is a disadvantage of such wetting solutions in that these contain ethoxylated emulsifiers, i.e. polyethylene glycol (PEG)-containing emulsifiers. Today, PEG-free emulsifier types that are as close as possible to natural emulsifiers are being demanded more and more frequently. In this connection, as PEG-free alternatives, for example, the following were proposed: in EP 1041391 the use of citric acid esters of monoglycerides and/or diglycerides of saturated fatty acids; in WO 02/056841 a combination of polyol poly(12-hydroxy stearate) and alkyl polyglycosides; and in EP 1700618 a combination of non-carbohydrate polyol partial esters and emulsifiers based on carbohydrates were proposed. However, even these mixtures are not yet satisfactory. The proposed citric acid esters are solid products and not easy to process. The alkyl polyglycosides are considered as disadvantageous due to their scouring effect on the skin. Other carbohydrate-based emulsifiers can only be prepared laboriously and are relatively expensive products.

Therefore, the object of the present invention is to provide wetting solutions for wet wipes that do not exhibit the disadvantages of known wetting solutions, which accordingly particularly do not contain any ethoxylated emulsifiers or emulsifiers with dermatologically questionable properties.

### DETAILED DESCRIPTION OF THE PREFERRED EMBODIMENTS

The set goal will be achieved by providing a wetting solution, containing:

a) at least one amino acid ester as an emulsifier

b) at least one oil or wax component and/or at least one further surfactant, and

c) water or an aqueous solution

The term “wetting solution” in the sense of the invention means real solutions but also emulsions of aqueous and oily phases. The emulsions can be oil-in-water emulsions (O/W) as well as water-in-oil emulsions. In many embodiments of the invention the “wetting solution” is in fact present as emulsion.

Preferred amino acid esters are esters of pyrrolidone carboxylic acid (PCA). As pyrrolidone carboxylic acid esters (PCA esters) the esters of pyrrolidone carboxylic acid and the fatty alcohols having 6-30 carbon atoms are advantageously used (C6-C30 alkyl PCA esters). Particularly advantageous are esters with 8-24 carbon atoms (CS-C24 alkyl PCA esters). Examples are esters with branched or unbranched alcohols, such as caproic alcohol, caprylic alcohol, 2-ethyl hexyl alcohol, nonanol, capric alcohol, undecyl alcohol, lauryl alcohol, tridecyl alcohol, myristyl alcohol, cetyl alcohol, palmoleyl alcohol, stearyl alcohol, isostearyl alcohol, oleyl alcohol, elaidyl alcohol, petroselinyl alcohol, linoleyl alcohol, linolenyl alcohol, arachyl alcohol, gadoleyl alcohol, behenyl alcohol, erucyl alcohol, bras [symbol] sidyl alcohol, and the Guerbet alcohols, preferably having 12-30 carbon atoms. All esters mentioned may also be used as mixtures thereof.

Therefore, the goal will be achieved by using esters based on amino acids, particularly esters of pyrrolidone carboxylic acid (PCA esters) alone or in a blend with one or more further surfactants, in order to stabilize wetting solutions for wet wipes. In principle, such esters are already known. As emulsifiers for stabilizing wetting solutions for wet wipes they have not yet been proposed.

Surprisingly, it was found in this connection that, as in the PIT process, emulsions can be prepared that are particularly finely dispersed and low-viscous. Such emulsions are particularly suitable for use as wetting solutions for wet wipes.

A particular advantage of these PCA esters used as emulsifiers is their improved skin compatibility as compared to ethoxylated emulsifiers, for example. This applies, for example, to the lauryl PCA ester, which exhibits a very low skin irritation potential. Pyrrolidone carboxylic acid itself is included as an ingredient in the skin's own NMF (natural moisturizing factor) and is already used as a moisturizer in cosmetic formulations.

A further advantage of wetting solutions or respective wet wipes, respectively, from the point of view of a manufacturer is the fact that many of the PCA esters are liquid, e.g. octyl PCA, decyl PCA and isostearyl PCA esters. These formulations can—in contrast to the PIT process—also be prepared inexpensively in a cold process. It is possible to avoid a complex formulation and preparation, as is necessary in many cases with respective wetting solutions and emulsions according to the prior art, e.g. when using the PIT process or a different process where compositions must be heated.

In the wetting solutions according to the invention the amino acid esters described above are included at a percentage of 0.05 to 20% by weight, more preferably at a percentage of 0.1 to 15% by weight, and particularly preferred at a percentage of 0.5 to 10% by weight.

The wetting solutions according to the present invention also contain at least one oil or wax component and/or at least one further surfactant. The composition of the wetting solution depends on the intended application area.

For example, it can be advantageous when cleaning wipes, such as eyeglass cleaning wipes, are free of oils or waxes, whereas wet wipes that are to have caring properties, such as facial cleaning wipes, make-up remover wipes or baby wipes, are desired to contain oil or wax components. In both cases, but particularly in the case of the cleaning wipes, the presence of one or more further surfactants is advantageous.

The oil or wax components are advantageously selected from the group of the branched or unbranched hydrocarbons. These include, for example, mineral oils, paraffin oil, paraffin wax, microcrystalline waxes, ozokerite, ceresine wax, squalane and squalene.

As natural oils it is possible to choose vegetable oils such as coconut oil, palm oil, palm kernel oil, olive oil, sunflower oil, soybean oil, peanut oil, rapeseed oil, castor oil, avocado oil, almond oil, Shea butter, sesame oil, wheat germ oil, rice bran oil, macadamia nut oil, lanolin, and neutral oil.

Furthermore, it is possible to advantageously choose the fatty alcohols having 14-30 carbon atoms, such as myristyl alcohol, cetyl alcohol, palmoleyl alcohol, stearyl alcohol, isostearyl alcohol, oleyl alcohol, elaidyl alcohol, petroselinyl alcohol, linoleyl alcohol, linolenyl alcohol, gadoleyl alcohol, behenyl alcohol, brassidyl alcohol, erucyl alcohol, and the Guerbet alcohols having 12-30 carbon atoms.

From the group of ester oils the oils of saturated and/or unsaturated, branched and/or unbranched alkane carboxylic acids with a chain length of 3-30 carbon atoms, and saturated and/or unsaturated, branched and/or unbranched alcohols with a chain length of 3-30 carbon atoms, from the group of aromatic carboxylic acids and saturated and/or unsaturated, branched and/or unbranched alcohols with a chain length of 3-30 carbon atoms. Such ester oils may be selected from the group containing isopropyl myristate, isopropyl palmitate, isopropyl stearate, isopropyl oleate, n-butyl stearate, n-hexyl laurate, n-decyl oleate, isooctyl step-arate [symbol] , isononyl stearate, 2-ethyl hexyl palmitate, 2-ethyl hexyl laurate, 2-ethy! hexyl isostearate, 2-ethyl hexyl cocoate, oleyl oleate, oleyl erucate, erucyl erucate, C12-15 alkyl benzoate, but also dicarboxylic acid esters, such as the tartrates or succinates of fatty alcohols having equal or different chain lengths, as well as synthetic and semi-synthetic and natural mixtures of such esters, such as jojoba oil and beeswax, carnauba wax or Candelilla wax. Furthermore, the esters of carbonic acid, such as 2-ethyl hexyl carbonate, dihexyl carbonate or dicapryl carbonate, or dialkyl ethers such as dioctyl ether, di-(2-ethyl hexyl)ether, dicapryl ether or didodecyl ether.

Advantageously, the oil phase may contain cyclic or linear silicone oils, or may completely consist of such oils. Advantageously, dimethicone or cyclomethicone is employed as silicone oil to be used according to the invention. However, other silicone oils may be used, such as hexamethyl cyclotrisiloxane, poly(dimethyl siloxane), poly(methyl phenyl siloxane), but also functionalized silicone oils.

The wetting solutions according to the invention contain up to 80% by weight of oil and/or wax components. The percentage of oil and/or wax components, if any, in the wetting solution is preferably 0.1 to 70% by weight. In many embodiments of the invention a percentage of 0.5 to 10% by weight is particularly pre [symbol] ferred.

The combination of pyrrolidone carboxylic acid esters (PCA esters) with further surfactants has also turned out to be advantageous. The term “surfactants” means boundary surface-active substances that form adsorption layers on surfaces or boundary surfaces or can aggregate by forming volume phases or lyotropic mesophases (Dorfler H 0, Grenzflachen und kolloid-disperse Systeme, Springer Berlin, Heidelberg, 2002). In this context these compounds are often low molecular compounds that contain a hydrophilic (polar) part and a hydrophobic (non-polar) part of mostly long-chain hydrocarbons. Here, a distinction is made between anion-active surfactants (anionic surfactants), cation-active surfactants (cationic surfactants), amphoteric surfactants (amphotensides), and non-ionic surfactants (niotensides). This classification principle is based on the charge structure of the head group.

Suitable for use as anionic surfactants in the preparation according to the invention, preferably in the form of sodium, potassium and ammonium salts or mono-, di- and trialkanole ammonium salts, as well as other counter ions such as basic amino acids, are the linear and branched fatty acids having 8 to 30 carbon atoms (soaps), such as lauric acid, aluminum stearate or zinc undecylenate; ether carboxylic acids, such as sodium laureth-13 carboxylate and sodium PEG-6 CQ-coamide carboxylate; amide ether carboxylic acids; alkyl sulfates such as lauryl sulfate; fatty alcohol ether sulfates, such as sodium laureth sulfate, sodium my [symbol] reth sulfate and sodium C12-C13-pareth sulfate; fatty acid ether sulfates; mono [symbol] glyceride(ether)sulfates such as sodium coco monoglyceride sulfate; alkyl benzene sulfonates; alkane sulfonates; olefin sulfonates; alkyl ether sulfonates; asulfo fatty acid methyl esters; sulfosuccinates, such as disodium lauryl sulfosuccinate, disodium laureth sulfosuccinate; succinamates; fatty acid isethionates such as sodium cocoyl isethionat; acyl lactylates, such as sodium or calcium stearoyl lactylate; alkyl(ether)tartrates; alkyl(ether)citrates, such as glyceryl stearate citrate and laureth-6 citrate; alkyl oligoglucoside sulfates and alkyl(ether)phosphates; and the acyl amino acids, such as acyl glutamate, cap [symbol] rylic/capric glutamate, palmitoyl aspartate, and their salts, but also other natural or non-natural amino acids; acyl-peptides, e.g. from wheat, soy, rice or milk protein or from collagen; finally sarcosinates, such as lauroyl sarcosine, cocoyl sarcosine, myristoyl sarcosine, and their salts; and taurates, such as sodium lauroyl tau rate or sodium methyl cocoyl tau rate.

Cationic surfactants to be used advantageously are alkyl amines, alkyl imidazoles, ethoxylated amines, quaternary surfactants, particularly the trialkyl ammonium chlorides and bromides, such as benzyl dimethyl stearyl ammonium chloride or cetyl trimethyl ammonium chloride or bromide, alkyl dimethyl hydroxyethyl ammonium chloride or bromide, dialkyl dimethyl ammonium chloride or bromide, dialkyl dimethyl ammonium chloride or bromide, dialkyl ethoxylate dimethyl ammonium chloride or bromide, alkyl pyridinium salts, imidazoline derivatives and esterquats. The amphoteric surfactants to be used include, for example, alkyl betaines; alkylamido betaines such as cocoamidopropyl betaine; alkyl ampho mono- and diacetates, such as sodium alkyl ampho acetate, disodium alkyl ampho diacetate; alkyl ampho mono- and ampho dipropionates; amino glycinates; sulfobetaines; amine oxides and phospholipids.

As additional non-ionic surfactants polyalkylene glycol ethers, esters or amides may be chosen. Such compounds are, for example, addition products of ethylene oxide and/or propylene oxide, with fatty alcohols, fatty acids, natural and non-natural fats and oils, fatty acid glycerides and fatty acid amides. Furthermore, esters that are formed by esterification of carboxylic acids with glycerol, polyglycerol, sorbitan, sugar or other alcohols, as well as their reaction products with alkylene oxides or alkanole amides, such as MEA, DEA or cocamide MIPA. Further, alkyl polyglycosides, such as lauryl glycoside, decyl glycoside and coco glycoside, or sugar surfactants of the fatty acid n-alkyl polyhydroxyl amides type.

The wetting solutions according to the invention contain up to 20% by weight of other surfactants. The percentage of further surfactants, if any, in the wetting solution is preferably 0.5 to 15% by weight and particularly preferred 1.0 to 10% by weight. In some embodiments of the invention the wetting solutions are free of further surfactants.

Advantageously, the wetting solution may contain typical cosmetic adjuvants, such as alcohols, particularly those having a low number of carbons, such as ethanol, isopropanol, dioJes or polyols having a low number of carbons, e.g. glycerol; the ethers of these alcohols, such as propylene glycol, ethylene glycol, ethylene glycol monoethyl or ethylene glycol monobutyl ether, propylene glycol monomethyl, propylene glycol monoethyl or propylene glycol monobutyl ether, diethylene glycol monomethyl, diethylene glycol monoethyl ether, diethylene glycol monobutyl ether and analogue products; polymers; foam stabilizers; electro [symbol] lytes; sugar derivatives; and moisturizers.

In certain embodiments glycerol is a preferred component of the wetting solutions or respectively the aqueous solution of the wetting solutions according to the invention. Preferably, it will then be employed in an amount of 0.5 to 8% by weight

In certain embodiments ethanol is a preferred component of the wetting solutions or respectively the aqueous solution of the wetting solutions according to the invention. Preferably, it will then be employed to the amount of up to 15% by weight.

Advantageous moisturizers are, for example, glycerol, propylene glycol, butylene glycol, hexylene glycol, sorbitol, sugar, lactic acid, amino acids, urea as described in “Skin Moisturization”, Ed. James J. Leyden, Anthony V. Rawlings, Marcel Dekker, 2002. Also applicable are polymeric moisturizers, such as waterswellable polysaccharides such as hyaluronic acid, chitosan or also fucosan-rich polysaccharides or peptides, proteins such as collagen.

The wetting solution may contain further active ingredients, additives and/or adjuvants, such as fragrances, e.g. perfume oil; thickeners; preservatives; antiirritation agents; antioxidants such as vitamin E; skin repair actives such as ceramides, peptides, etc.; anti-wrinkle agents; anti-acne agents; self-tanning agents; or sunscreens.

Suitable as preservatives are, for example, phenoxy ethanol, parabenes, benzoic acid, sorbic acid, dehydroacetic acid, ethyl hexyl glycerol and the other substances described in the EU Cosmetic Directive, Annex 6.

The wetting solutions of the invention contain at least 10% by weight of water or of an aqueous solution, wherein “aqueous solution” means a solution that consists predominantly of water.

The wetting solution in the sense of the present invention can have a low viscosity, i.e. up to 2,000 mPas, or can be present in a viscous form, as a cream or in a wax-like form. In particular embodiments of the invention it can also be present as a gel. Preferably, it has a low viscosity of up to 2,000 mPas.

Different types of wipes or tissues, respectively, can be wetted with the wetting solution described above in order to obtain the wet wipes according to the invention. These can then especially be used for cosmetic, body care, dermatologica! and/or cleaning purposes.

The terms “wipe” and “other flexible material” in the sense of the present invention mean all kinds of woven or non-woven flexible materials that may be wetted with the wetting solutions described above. Particularly, woven or non-woven wipes, sponges, cotton batting-like materials and the like are meant. All kinds of natural and/or synthetic fibers may be considered as materials. In the case of sponges, natural or synthetic sponges may be considered.

Examples of wetted wipes or materials in the sense of the present invention are cleaning wipes, facial cleaning wipes, make-up remover wipes. refreshment wipes, baby wipes, wet toilet paper, eyeglass cleaning wipes, and other wet wipes.

Also an object of the present invention is a concentrate for preparing the wetting solution described above, wherein the concentrate contains: a) at least one of the amino acid esters described above as an emulsifier, and b) at least one of the oil or wax components described above and/or at least one of the further surfactants described above.

### Example 1

### Wax-Like Wetting Solution

The components of Phase A are melted at aG° C. and stirred until homogeneous. The water is heated to ao° c. and then added to the homogeneous Phase A. Homogenization is performed by using an Ultra-Turrax, followed by cooling down. A solid emulsion is obtained.

| Example 1 | Ingredient | % by weight |
|---|---|---|
| A | Olive oil/isopropyl palmitate | 60 |
| | Isostearyl PCA | 8 |
| | Candelilla wax | 7 |
| B | Water | Ad 100 |

The wax-like wetting solution may subsequently be melted by heating and be sprayed hot onto paper wipes. The paper wipes wetted this way can be used, for example, for hygiene paper products, such as handkerchiefs or toilet paper.

### Examples 2 to 7

### Wetting Solutions Containing Oil

The components of Phase A are mixed in the given order at room temperature until homogeneous. Afterwards, the water, or Phase S, respectively, is slowly added while stirring. Subsequently the pH is set to 5.0-7.0 using a diluted citric acid solution and/or a diluted sodium hydroxide solution.

| Example 2 | Ingredient | % by weight |
|---|---|---|
| A | Paraffinum perliquidum | 5.0 |
| | Isostearyl PCA | 2.0 |
| | Sodium cocoyl glutamate | 5.0 |
| | Perfume oil | 0.1 |
| | Preservatives (Phenoxy ethanol, benzoic acid, | 0.5 |
| | dehydroacetic acid, and ethyl hexyl glycerol) | |
| B | Glycerol | 1.0 |
| | Water | Ad 100 |

A finely dispersed, slightly opalescent emulsion is obtained.

| Example 3 | Ingredient | % by weight |
|---|---|---|
| A | Paraffinum perliquidum | 1.5 |
| | Dodecyl hexadecyl PCA | 0.2 |
| | Isostearyl PCA | 0.5 |
| | Sodium cocoyl glutamate | 1.9 |
| | Perfume oil | 0.1 |
| | Phenoxy ethanol, benzyl alcohol, potassium | 0.5 |
| | sorbate, tocopherol | |
| B | Disodium laureth | 2.5 |
| | Water | Ad 100 |

A finely dispersed, almost clear emulsion is obtained.

| Example 4 | Ingredient | % by weight |
|---|---|---|
| A | Silicone oil 200/200 cs | 1.0 |
| | Isostearyl PCA | 1.0 |
| | Octyl PCA | 0.5 |
| | Cocoamidopropyl betaine | 2.5 |
| | Perfume oil | 0.1 |
| | Phenoxy ethanol, methyl, butyl, thyl, propyl | 0.5 |
| | and isobutyl parabene | |
| B | Water | Ad 100 |

A finely dispersed opalescent emulsion is obtained.

| Example 5 | Ingredient | % by weight |
|---|---|---|
| A | C12-15 alkyl benzoate | 3.0 |
| | Decyl PCA | 1.4 |
| | Octyl PCA | 1.4 |
| | Sodium cacoyl glutamate | 7.6 |
| | Perfume oil | 0.1 |
| | Phenoxy ethanol, benzoic acid, dehydroacetic | 0.75 |
| | acid, ethyl hexyl glycerol | |
| B | Cocoamidopropyl betaine | 7.5 |
| | Glycerol | 1.0 |
| | Water | Ad 100 |

A finely dispersed, almost clear emulsion is obtained.

| Example 6 | Ingredient | % by weight |
|---|---|---|
| A | Isopropyl palmitate | 7.0 |
| | Isostearyl PCA | 1.0 |
| | Decyl PCA | 0.5 |
| | Sodium cacoyl glutamate | 3.4 |
| | Perfume oil | 0.1 |
| | Phenoxy ethanol, methyl, butyl, ethyl, | 0.5 |
| | propyl and isobutyl parabene | |
| B | Glycerol | 1.0 |
| | Water | Ad 100 |

A finely dispersed white emulsion is obtained.

| Example 7 | Ingredient | % by weight |
|---|---|---|
| A | Dicapryl ether | 5 |
| | Octyl PCA | 3 |
| | Sodium laureth sulfate | 4 |
| | Perfume oil | 0.1 |
| | Phenoxy ethanol, methyl, butyl, ethyl, | 0.5 |
| | propyl and isobutyl parabene | |
| B | Glycerol | 1.0 |
| | Water | Ad 100 |

A finely dispersed, almost transparent emulsion is obtained

The wetting solutions of Examples 2 to 7 can be used, for example, for wet wipes for hand cleaning or skin care.

### Example 8

### Aqueous Wetting Solution

The components of Phase A and Phase B are mixed separately. Afterwards, Phase B is slowly added to Phase A while stirring. Subsequently the pH is set to 5.0-7.0. A clear-looking solution (micellar solution) is obtained.

| Example 8 | Ingredient | % by weight |
|---|---|---|
| A | Isostearyl PCA | 0.5 |
| | Sodium cocoyl glutamate | 1.4 |
| | Prefume oil | 0.1 |
| | Phenoxy ethanol, methyl, butyl, ethyl, | 0.5 |
| | propyl and isobutyl parabene | |
| B | Cocoamidopropyl betaine | 7.5 |
| | Glycerol | 2.0 |
| | Water | Ad 100 |

This wetting solution can be used, for example, for wet wipes for cleaning purposes or for eyeglass cleaning wipes.

### Example 9

### Alcoholic Wetting Solution

The components of Phase A are mixed in the given order until homogeneous. Afterwards, the water is added slowly. Finally, the ethanol is added while stirring. Subsequently, the pH is set to 5.0-7.0. An opalescent solution (micellar solution) is obtained.

| Example 9 | Ingredient | % by weight |
|---|---|---|
| A | Isostearyl PCA | 1.0 |
| | Sodium lauroyl sarcosinate | 3.0 |
| | Perfume oil | 0.1 |
| | Phenoxy ethanol, methyl, butyl, ethyl, | 0.5 |
| | propyl and isobutyl parabene | |
| B | Ethanol | 8.0 |
| | Water | Ad 100 |

This wetting solution can be used, for example, for cleaning purposes or for eyeglass cleaning wipes.

What is claimed is:

1. A wetting solution for manufacturing wet wipes or other wet flexible materials for cosmetic, body care, dermatological and/or cleaning purposes, containing:
a) at least one amino acid ester as an emulsifier
b) at least one oil or wax component and/or at least one further surfactant, and
c) water or an aqueous solution

2. The wetting solution according to claim 1, wherein said amino acid ester(s) are esters of pyrrolidone carboxylic acid (PCA).

3. The wetting solution according to claim 2, wherein said PCA ester(s) are C₁-C₃₀ alkyl PCA esters.

4. The wetting solution according to claim 3, wherein said ester(s) are selected from the group comprising the PCA esters of caproic alcohol, caprylic alcohol, 2-ethyl hexyl alcohol, nonanol, capric alcohol, undecyl alcohol, lauryl alcohol, tridecyl alcohol, myristyl alcohol, cetyl alcohol, palmoleyl alcohol, stearyl alcohol, isostearyl alcohol, oleyl alcohol, elaidyl alcohol, petroselinyl alcohol, linoleyl alcohol, linolenyl alcohol, arachyl alcohol, gadoleyl alcohol, behenyl alcohol, erucyl alcohol, brassidyl alcohol, and the Guerbet alcohols, or mixtures thereof.

5. The wetting solution according to claim 1, wherein said at least one oil or wax component is selected from the group comprising branched or unbranched hydrocarbons, particularly mineral oils, paraffin oil, paraffin wax, microcrystalline oils, ozokerite, ceresine wax, squalane and squalene; natural oils, particularly coconut oil, palm oil, palm kernel oil, olive oil, sunflower oil, soybean oil, peanut oil, rapeseed oil, castor oil, avocado oil, almond oil, Shea butter, sesame oil, wheat germ oil, rice bran oil, macadamia nut oil, lanolin, and neutral oil; fatty alcohols having 14-30 carbon atoms, particularly myristyl alcohol, cetyl alcohol, palmoleyl alcohol, stearyl alcohol, isostearyl alcohol, oleyl alcohol, elaidyl alcohol, petroselinyl alcohol, Iinoleyl alcohol, linolenyl alcohol, gadoleyl alcohol, behenyl alcohol, bras-sidyl alcohol, and erucyl alcohol; Guerbet alcohols having 12-32 carbon atoms; ester oils, particularly isopropyl myristate, isopropyl palmitate, isopropyl stearate, isopropyl oleate, n-butyl stearate, n-hexyl laurate, n-decyl oleate, isooctyl stearate, isononyl stearate, 2-ethyl hexyl palmitate, 2-ethyl hexyl laurate, 2-ethyl hexyl isostearate, 2-ethyl hexyl cocoate, oleyl oleate, oleyl erucate, erucyl erucate, C12-15 alkyl benzoate, and tartrates or succinates of fatty alcohols having equal or different chain lengths; as well as synthetic and semi-synthetic and natural mixtures of such esters, particularly jojoba oil, beeswax, carnauba wax or CandeJilla wax; esters of carbonic acid, particularly 2-ethyl hexyl carbonate, dihexyl carbonate or dicapryl carbonate, dialkyl ethers, particularly dioctyl ether, di-(2-ethyl hexyl)ether, dicapryl ether or didodecyl ether; silicone oils, particularly dimethicone or cyclomethicone, hexamethyl cyclotrisiloxane, poly(dimethyl siloxane) and poly(methyl phenyl siloxane); or mixtures of the aforementioned oils or waxes, respectively.

6. The wetting solution according to claim 1, wherein said wetting solution additionally contains one or more surfactants from the group of anionic surfactants, cationic surfactants, amphoteric surfactants and non-ionic surfactants, or mixtures thereof.

7. The wetting solution according to claim 6, wherein said anionic surfactant is selected from the group consisting of: a linear or branched fatty acid having 8 to 30 carbon atoms (soaps); an ether carboxylic acid; an amide ether carboxylic acid; an alkyl sulfate; a fatty alcohol ether sulfate; a fatty acid ether sulfate; a monoglyceride(ether)sulfate; an alkyl benzene sulfonate; an alkane sulfonate; an olefin sulfonate; an alkyl ether sulfonate; a-sulfo fatty acid methyl ester; a sulfosuccinate; a succinamate; a fatty acid isethionate; an acyl lactylate; an alkyl(ether)tartrate; an alkyl(ether)citrate; an alkyl oligoglucoside sulfate or an alkyl(ether)phosphate; an acyl amino acid; an acylwpeptide; a sarcosinate; a taurate; preferably an acyl amino acid; an acyl-peptide, a sarcosinate, a taurate; a salt of the aforementioned compounds, or a mixture thereof.

8. The wetting solution according to claim 1, wherein said wetting solution or said aqueous solution contains one or more further components, selected from the group comprising alcohols, preferably ethanol, isopropanol; diols or polyols such as glycerol; ethers, preferably propylene glycol, ethylene glycol, ethylene glycol monoethyl or ethylene glycol monobutyl ether, propylene glycol monomethyl, propylene glycol monoethyl, propylene glycol monobutyl ether, diethylene glycol monomethyl, diethylene glycol monoethyl ether and diethylene glycol monobutyl ether; as well as polymers, foam stabilizers; electrolytes, sugar derivatives and moisturizers.

9. The wetting solution according to claim 1, wherein said wetting solution or said aqueous solution comprises one or more further active ingredients, additives and/or adjuvants, selected from the group comprising fragrances; thickeners; preservatives; anti-irritation agents; antioxidants; skin repair actives; anti-wrinkle agents; anti-acne agents; self-tanning agents; or sunscreens.

10. The wetting solution according to claim 1, wherein said wetting solution has a low viscosity, a high viscosity, is a cream or a gel.

11. A wet wipe or other wet flexible material for cosmetic, body care, dermatological and/or cleaning purposes, wherein said wipe or said material is wetted with a wetting solution according to claim 1.

12. The wet wipe or material according to claim 11, wherein said wipe consists of natural and/or synthetic fibers.

13. The wet wipe or material according to claim 11, wherein said wetted wipe or material is selected from the group comprising cleaning wipes, facial cleaning wipes, make-up remover wipes, refreshment wipes, baby wipes, wet toilet paper, eyeglass cleaning wipes and other wet wipes.

14. Wet wipes or wet flexible materials comprising amino acid esters, as emulsifiers in wetting.

15. A concentrate for preparing a wetting solution according to claim 1, containing:
a) at least one amino acid ester as an emulsifier, and
b) at least one oil or wax component and/or at least one further surfactant.

16. A method for manufacturing a wetting solution according to claim 1, containing the steps:
a) stirring of all components, beside the water or the components of the aqueous solution, respectively, until homogeneous in order to obtain a Phase A,
b) adding the water or the aqueous solution, respectively, to Phase A,
c) homogenizing, and afterwards
d) setting the pH value to 5.0 to 7.0, preferably with diluted citric acid solution and/or diluted sodium hydroxide solution.

17. The method according to claim 16, wherein in step a) the components are melted before and/or while stirring until homogeneous, in step b) the water or the aqueous solution, respectively, is heated prior to adding it to Phase A up to a temperature that corresponds to the temperature of Phase A +/−5° C., and, optionally, after step d) cooling down the completed wetting solution respectively allowing it to cool down.

18. The wetting solution according to claim 3, wherein said PCA ester(s) are C₆-C₃₀ alkyl PCA esters.

19. The wetting solution according to claim 18, wherein said PCA ester(s) are C₈-C₂₄ alkyl PCA esters.

20. Wet wipes or wet flexible materials of claim 13 comprising esters of pyrrolidone carboxylic acid, as emulsifiers in wetting.

21. A method for manufacturing a wetting solution according to claim 16, wherein the step of setting the pH value to 5.0 to 7.0 is performed with diluted citric acid solution and/or diluted sodium hydroxide solution.

22. The method according to claim 17, further comprising after step d) cooling down the completed wetting solution respectively allowing it to cool down.
